(19) United States
(12) Patent Application Publication
(10) Pub. No.: US 2007/0191842 A1
(43) Pub. Date: Aug. 16, 2007

(54) Spinal fixation devices and methods of use

(75) Inventors: Fred J. Molz IV, Birmingham, AL (US); Jonathan M. Dewey, Memphis, TN (US)

Correspondence Address: COATS & BENNETT, PLLC, 1400 Crescent Green, Suite 300, Cary, NC, 27518, US

(73) Assignee: SDGI Holdings, Inc.

(21) Appl. No.: 11/343,157

(22) Filed: Jan. 30, 2006

(51) Int. Cl. A61F 2/30

(52) U.S. Cl. 606/61

(57) ABSTRACT

A spinal fixation device comprises a receiver that can be used with rods of different sizes. The receiver comprises a body with a channel formed therein. The body is adapted to receive one of a set of interchangeable inserts that engage rods of different sizes. A bone anchor secures the receiver to a vertebral member.

### BACKGROUND

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve.

Vertebral rods may be implanted to redistribute stresses and/or restore proper alignment of the vertebral members in one or more of these regions. The rods extend along a section of the spine and may include a curved configuration to conform to the curvature of the spine. Often times two or more rods are connected together and work in combination to support and position the vertebral member.

Typically, different anchoring hardware is used to secure rods of different sizes to the spinal column. It would be useful to have anchoring hardware for use with rods of different sizes.

### SUMMARY

The present application is directed to devices and methods for attaching a rod to a vertebral member. One device comprises a receiver that can be used with rods of different sizes. The receiver may comprise a body with a channel formed therein to receive a rod. The body may be adapted to receive one of a set of interchangeable inserts that engage rods of different sizes. A bone anchor may secure the receiver to a vertebral member. The receiver and bone anchor may be integrally formed, or may be separate elements.

### BRIEF DESCRIPTION OF THE DRAWINGS

FIG. 1 is a perspective view illustrating an embodiment of a spinal fixation device comprising a receiver, insert, locking member and bone anchor.

FIG. 2 is a side elevation view of one embodiment.

FIG. 3 is a top view of one embodiment.

FIGS. 4A-4D illustrate various alternative configurations for the insert.

FIG. 5 illustrates a composite insert.

FIG. 6 illustrates an insert with integral retaining features to prevent longitudinal movement of the insert in the receiver.

FIG. 7 illustrates the insert of FIG. 6 engaged with a receiver.

FIG. 8 illustrates an insert with orientation features to orient the insert in a predetermined orientation.

FIG. 9 is a perspective view illustrating an embodiment of the spinal fixation device.

FIG. 10 is an elevation view illustrating one embodiment.

FIG. 11 illustrates is a perspective view illustrating one embodiment of the spinal fixation device.

FIG. 12 illustrates is a perspective view illustrating one embodiment of the spinal fixation device.

### DETAILED DESCRIPTION

The present application is directed to devices and methods for attaching a rod to a vertebral member. The devices and methods provide common hardware for anchoring rods of different sizes to a vertebral member. One device comprises a receiver having a channel formed therein to receive one of a set of interchangeable inserts that engage rods of different sizes.

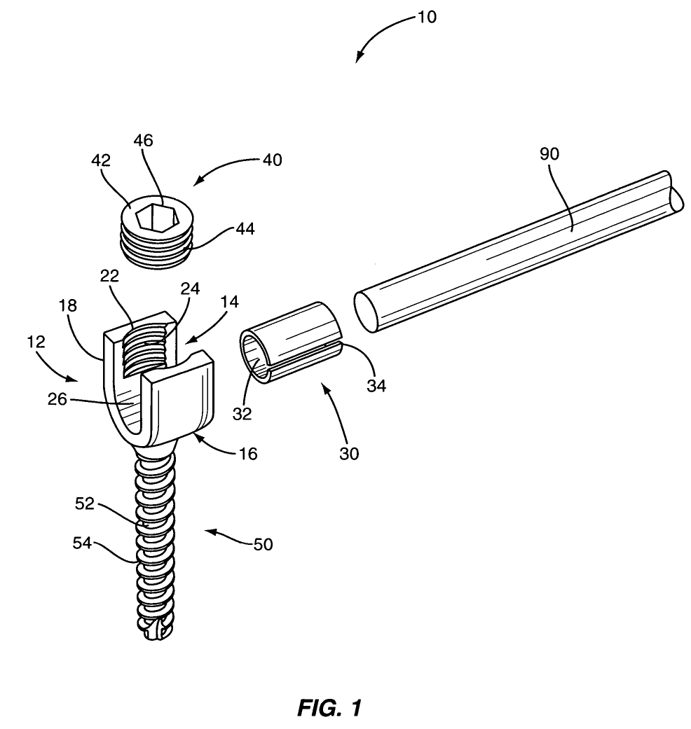

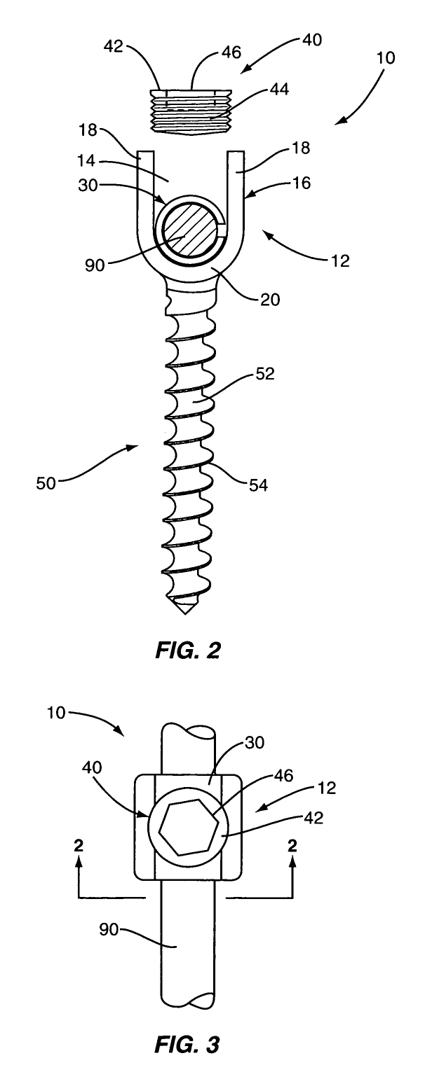

Referring now to the drawings, FIGS. 1-3 illustrate one embodiment of a spinal fixation device, indicated generally by the numeral 10, for attaching a rod 90 to a patient's spine. The spinal fixation device 10 comprises a receiver 12 including a channel 14 for accepting a rod 90, an insert 30 disposed around the rod 90, a locking member 40 to lock the rod 90 in the channel 14 of the receiver 12, and a bone anchor 50 to fix the receiver 12 to a vertebral member.

In one embodiment, the receiver 12 comprises a generally u-shaped body 16 with opposing sidewalls 18 connected by an arcuate lower section 20. The interior of the sidewalls 18 include concave surfaces 22 with threads 24 formed therein to engage the locking member 40 as will be hereinafter described. The lower portion of the channel 14 is generally circular and forms a seat 26 to support the rod 90.

In one embodiment, the insert 30 is generally cylindrical in shape and has an axial bore 32 sized to fit a rod 90 of predetermined size. The outer diameter of the insert 30 is sized to conform to the curvature of the channel 14. In one exemplary embodiment, a plurality of similar inserts 30 are provided with the same outer diameter but with different bore sizes to accommodate rods 100 of different sizes. Having a plurality of inserts 18 allows the same receiver 12 to be used for rods 100 of two or more different sizes. The cylindrical insert 30 may further include a slot 34 that extends the length of the insert 30. The slot 34 allows the insert 30 to be crimped to firmly secure the insert 30 to the rod 90.

The locking member 40 comprises a locking screw 42 with external threads 44 that engage corresponding threads 24 formed on the concave surfaces 22 of the receiver 12. The locking screw 42 further includes a hexagonal socket 46 to receive a wrench. When inserted into the receiver 12 and tightened, the locking screw 42 engages and compresses the insert 30. The compression force generated by the locking screw 42 crimps the insert 30 to firmly secure the insert 30 to the rod 90. Additionally, the compression forces secure the rod 90 within the channel 14 of the receiver 12. In one embodiment, the insert 30 includes a geometry that can collapse to a certain point and apply a predetermined force to the rod 90 while transferring the remaining force through to the channel 14. In one specific embodiment, the insert 30 is designed such that it collapses to a continuous “O” shape that conforms to the shape of the rod 90.

The bone anchor 50 comprises a bone screw 52, which in this embodiment is integrally formed with the receiver 12. In other embodiments, some of which are described below, the bone screw 52 may comprise a separate element. The bone screw 52 includes threads 54 that are configured to grip bone.

In use, the surgeon places two or more spinal fixation devices 10 along the patient's spine with the channels 14 vertically aligned and the bone screw 52 secured within the vertebral member. Once the receivers 12 are positioned, the inserts 30 are fit over the rod 90 and the rod 90 and inserts 30 are inserted into the aligned channels 14 of the receivers 12. The locking screws 42 are then inserted and tightened against the inserts 30. Tightening the locking screws 42 compresses the inserts 30 to lock the inserts 30 to the rod 90 and to firmly secure the rod 90 within the channels 14 of the receivers 12. In certain cases, the inserts 30 may also act as a force-distributing or force-shielding buffer between the locking screw 42 and the rod 90.

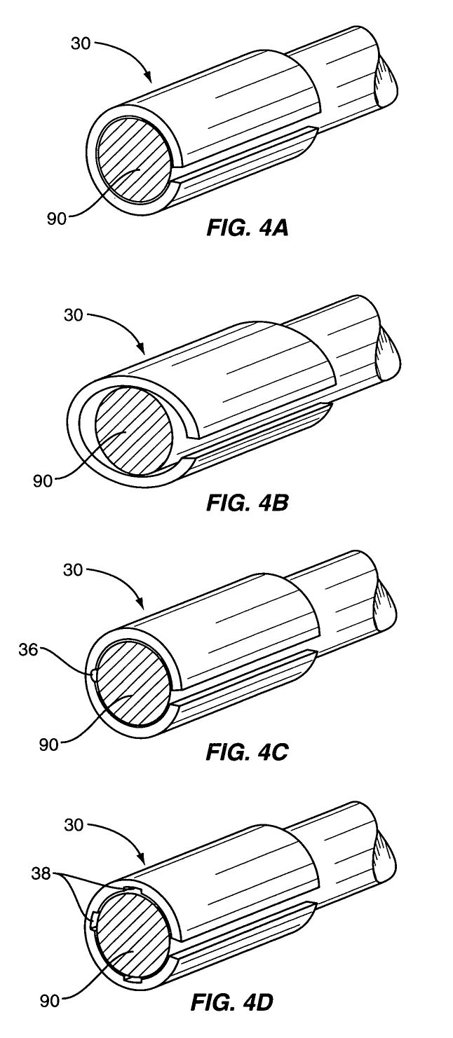

FIGS. 4A-4B illustrate alternative configurations of the insert 30 for various embodiments of the spinal fixation device 10. In FIG. 4A, the insert 30 and rod 90 have congruent circular shapes, though other shapes could also be used. In other embodiments, the shape of the insert 30 may be different than the shape of the rod 90 as shown in FIG. 4B. In FIG. 4B, the insert 30 has an oval shape and the rod 90 has a circular shape. The insert 30 may also include features, such as notches 36 (FIG. 4C) and cut-outs 38 (FIG. 4D) to facilitate crimping or collapsing of the insert 30 and/or to focus clamping forces.

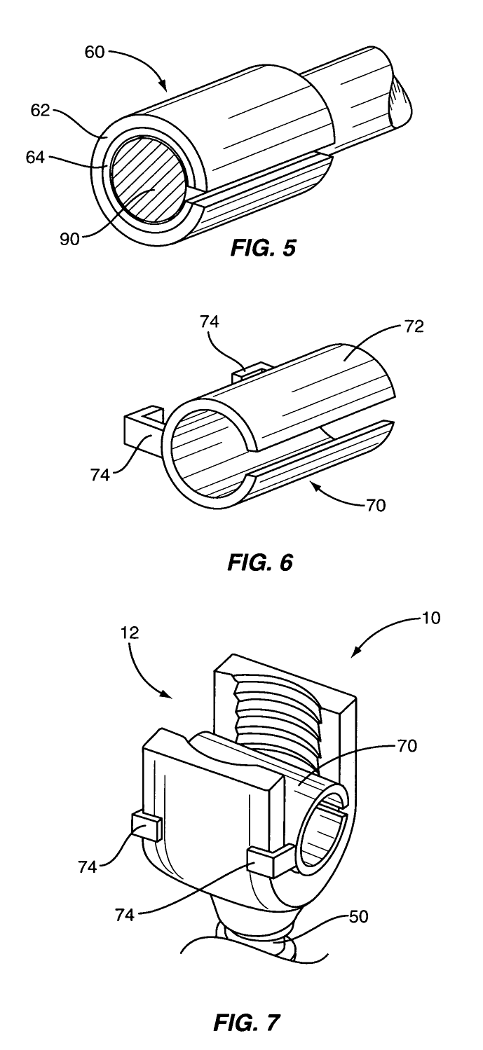

FIG. 5 illustrates an exemplary composite insert 60 made using two different materials for outer and inner portions 62, 64. In one embodiment, the composite insert 60 comprises an outer portion 62 made of a rigid or semi-rigid material, and in inner portion 64 made of a material with a relatively low hardness. The outer portion 62 may, for example, be made of a metal or a ceramic. The inner portion 64 may be made of polymer material. Any known methods of fixing the inner and outer portions together may be used including integral molding, mechanical interlocks (e.g. snap features), gluing, threading, pinning, etc. The rigid outer portion 62 is engaged by the locking screw 42 and transfers compressive forces to the inner portion 64. The inner portion 64 transfers compressive forces to the rod 90 without scratching or marring the rod 90. In one embodiment, the hardness of the inner portion 64 is less than the hardness of the rod 90 to which it is attached.

FIGS. 6 and 7 illustrate an exemplary insert 70 with a retaining feature to help retain the insert 70 within channel 14. In one embodiment, the insert 70 comprises a cylindrical body 72 as previously described with retaining features at opposing ends thereof. The retaining features comprise small hooks 74 that wrap around the sidewall 18 of the receiver 12 to prevent or reduce longitudinal movement of the insert 30 within the channel 14. Other types of retaining features could also be used. For example, a key or pin could be used instead of hooks 74 to prevent longitudinal movement of the insert 70 within the channel 14.

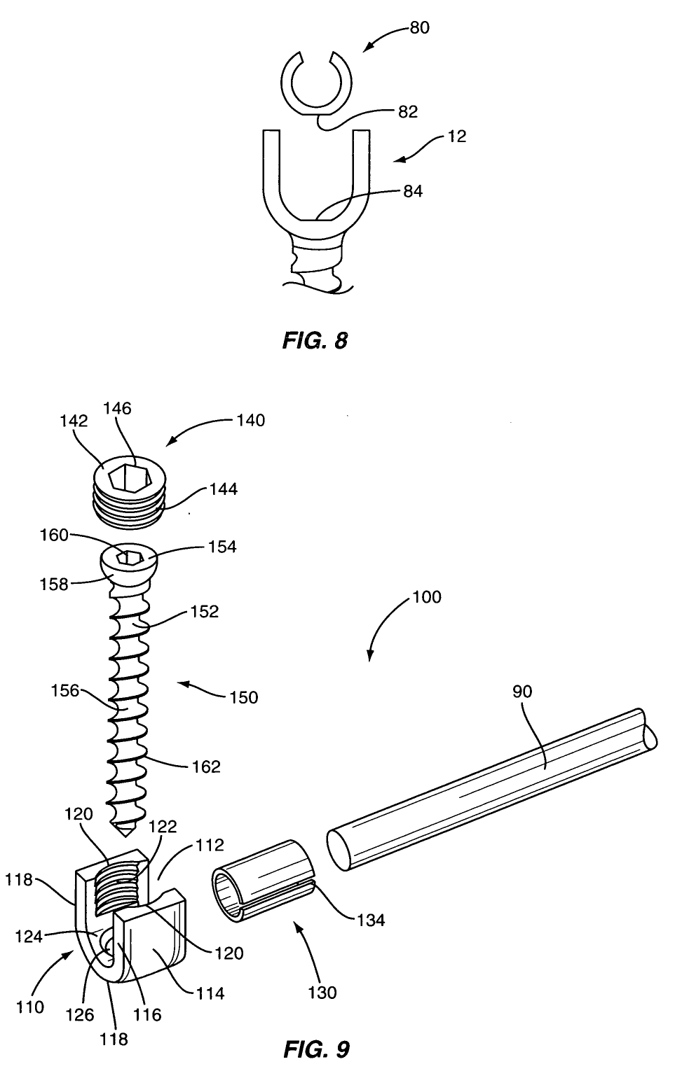

FIG. 8 illustrates an exemplary insert 80 having an orientation feature to ensure orientation of the insert 30 in a desired position. In this embodiment, the receiver 12 and insert 30 have complementary features that provide a desired orientation for the insert 30. More particularly, the insert 80 is formed with a flat 82 in the outer surface thereof which contacts a corresponding flat 84 in the channel 14. The engagement of the flat 82 on the outer surface of the insert 80 with the flat 84 in the channel 14 orients the insert 80 in a predetermined orientation.

In one embodiment as illustrated in FIG. 8, the insert 80 includes an opening that is substantially aligned with the opening in the receiver 12. This allows for insertion of the rod 90 in a similar manner as if the insert 80 was not within the receiver and no additional or tertiary steps are necessary for positioning the rod 90 within the insert 80.

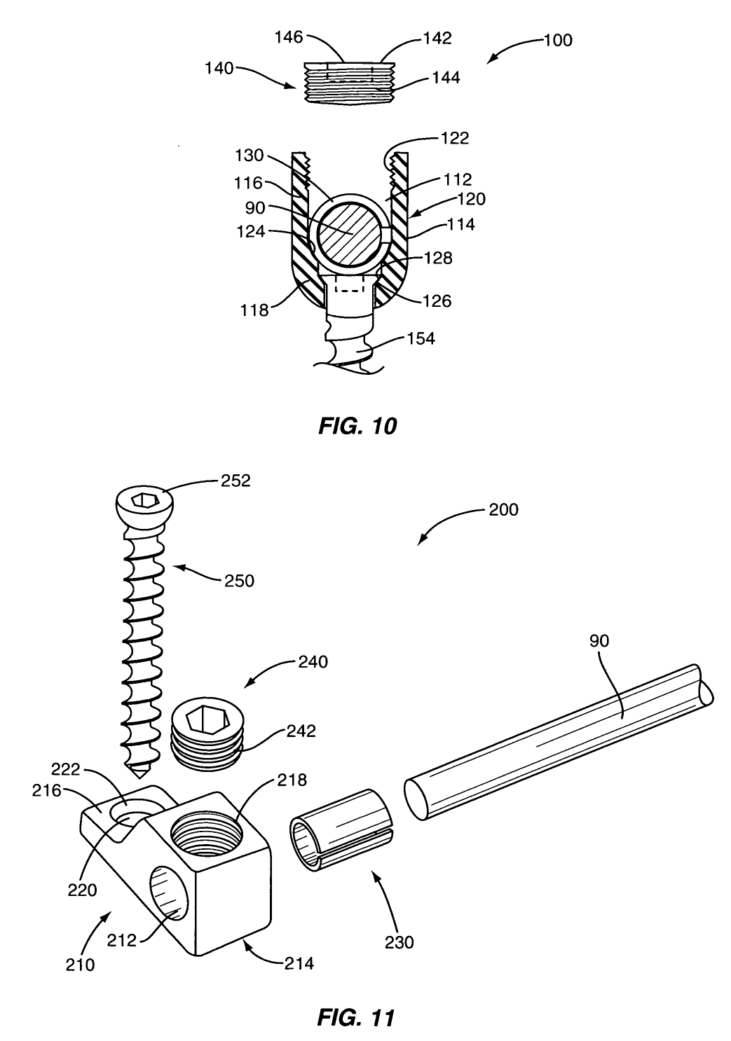

FIGS. 9 and 10 illustrate another embodiment of a spinal fixation device, indicated generally by the numeral 100. The spinal fixation device 100 comprises a receiver 110 including a channel 112 for accepting a rod 90, an insert 130 disposed around the rod 90, a locking member 140 to lock the rod 90 in the channel 112 of the receiver 110, and a bone anchor 150 to fix the receiver 110 to a vertebral member.

In one embodiment, the receiver 110 comprises a generally u-shaped body 114 with opposing sidewalls 116 connected by an arcuate lower section 118. The interior of the sidewalls 116 include concave surfaces 120 with threads 122 formed therein to engage the locking member 130 as will be hereinafter described. The lower portion of the channel 112 is generally circular and forms a seat 124 to support the rod 90. A screw hole 126 is formed in the lower section 118 of the body that intersects the channel 112. The screw hole 126 includes a spherical seat 128 and is configured to receive the bone anchor 150 as will hereinafter be described.

The insert 130 is generally cylindrical in shape and has an axial bore 132 sized to fit a rod 90 of predetermined size. The outer diameter of the insert 130 is sized to conform to the curvature of the channel 112. As previously described, a plurality of similar inserts 130 with the same outer diameter but with different bore sizes may be provided to accommodate rods 100 of different sizes. Having a plurality of inserts 130 allows the same receiver 110 to be used with rods 12 of two or more different sizes. The cylindrical insert 130 further includes a slot 134 that extends the length of the insert 130. The slot 134 allows the insert 130 to be crimped to firmly secure the insert 130 to the rod 90.

The locking member 140 comprises a locking screw 142 with external threads 144 that engage corresponding threads 122 formed on the concave surfaces 120 of the receiver 110. The locking screw 142 further includes a hexagonal socket 146 to receive a wrench. When inserted into the receiver 110 and tightened, the locking screw 142 engages and compresses the insert 130. The compression force generated by the locking screw 142 crimps the insert 130 to firmly secure the insert 130 to the rod 90. Additionally, the compression forces secure the rod 90 within the channel 112 of the receiver 110 to prevent movement of the rod 90.

The bone anchor 150 comprises a bone screw 152 having a head 154 and a threaded shank 156. The head 154 has a spherical outer surface 158 and a socket 160 to receive a wrench. The spherical outer surface 158 contacts the spherical seat 128 of the screw hole 126 as best seen in FIG. 10. The spherical surface 158 and spherical seat 128 allow the angle of the receiver 120 to be adjusted about three axis of rotation relative to the axis of the bone screw 152. The shank 156 includes threads 162 configured for gripping bone.

In use, the surgeon fixes two or more receivers 110 to the spinal column of the patient. The bone screws 152 are inserted through the screw holes 156 in the lower portions of respective receivers 110 and driven into the respective vertebral members. Before fully tightening the bone screw 152, the angular position of the receiver 110 may be adjusted to properly orient and align the channels 112 in the receivers 110. Once the receivers 110 are anchored in place, the inserts 130 are fit over the rod 90 and the rod 90 and inserts 130 are inserted into the aligned channels 112 of the receivers 12. The locking screws 142 are then inserted and tightened against the inserts 130. Tightening the locking screws 42 compresses the inserts 130 to lock the inserts 130 to the rod 90, and to firmly secure the rod 90 within the channels 112 of the receivers 110.

FIG. 11 illustrates another embodiment of the spinal fixation device indicated generally by the numeral 200. The spinal fixation device 200 comprises a receiver 210 including a channel 212 for accepting a rod 90, an insert 230 disposed around the rod 90, a locking member 240 to lock the rod 90 in the channel 212 of the receiver 210, and a bone anchor 250 to fix the receiver 210 to a vertebral member. In this embodiment, the insert 230, locking member 240, and bone anchor 250 are the same as described in the previous embodiment.

The receiver 210 in this embodiment comprises a mounting block 214 having a tongue section 216. A channel 212 to receive the insert 230 is formed in the mounting block 214. A screw hole 218 configured to receive the locking screw 242 intersects with the channel 212. Insert 230 fits within the channel 212 and the rod 90 passes through the insert 230. Tightening the locking screw 242 against the insert 230 locks the insert 230 to the rod 90 and secures the rod 90 within the channel 212 of the mounting block 214 as previously described. The tongue portion 216 includes a screw hole 220 configured to receive the bone screw 250. Screw hole 220 may include a spherical seat 222 that contacts the head 252 of the bone screw 250. The bone screw 250 passes through the screw hole 220 and penetrates the vertebral member to secure the mounting block 214 to the vertebral member. The spherical surface of the screw head 252 allows for some angular adjustment of the mounting block 214 before the bone screw 250 is fully tightened.

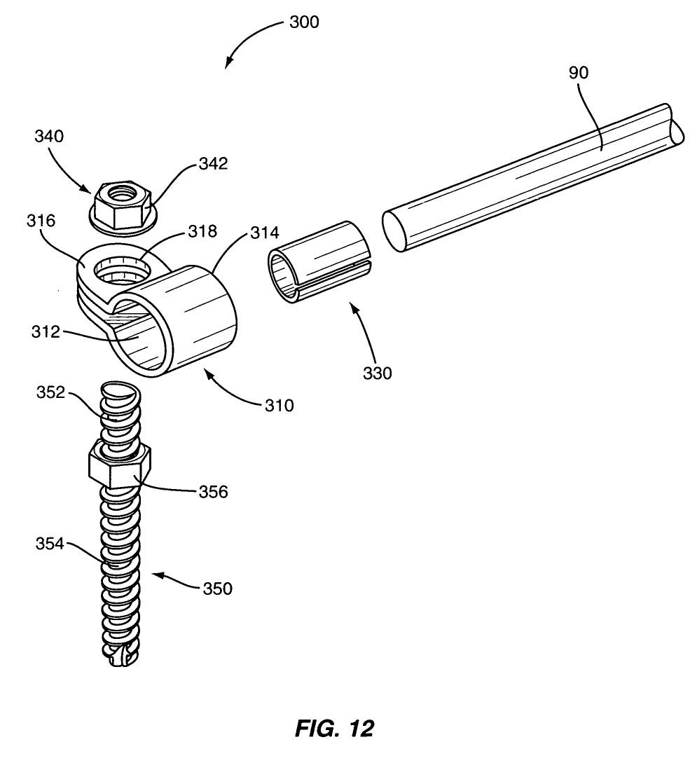

FIG. 12 one embodiment of the spinal fixation device indicated generally by the numeral 300. The spinal fixation device 300 comprises a receiver 310, including a channel 312 for accepting a rod 90, and insert 330 disposed around the rod 90, a locking member 340 to lock the rod 90 in the channel 312, and a bone anchor 350 to fix the receiver 310 to a vertebral member. In this embodiment, the insert 330 is the same as the previous embodiments. The receiver 310, locking member 340, and bone anchor 350, however, are different.

The receiver 310 in this embodiment comprises a C-shaped clamp 314 having a tongue 316. The C-shaped clamp 314 defines a channel 312 to receive the insert 330. The tongue 316 of the C-shaped clamp 314 includes an opening 318 to receive an upper portion of the bone screw 350. The bone screw 350 includes a shank having an upper portion 352 and lower portion 354 separated by a fixed nut 356. The upper portion 352 of the bone screw 350 is configured to pass through the hole 318 in the C-shaped clamp 314. Locking member 340 comprises a lock nut 342 that threads onto the upper portion 352 of the bone screw 350. Tightening the lock nut 342 tightens the C-shaped clamp 314 around the insert 330. The compressive forces so generated lock the insert 330 to the rod 90 and secure the rod 90 in the channel 312. The lower portion 354 of the bone screw 350 includes threads adapted to grip bone. Integral nut 356 can be engaged by a wrench (not shown) to provide rotational force for driving the bone screw 350 into a vertebral member.

In the embodiments described above, inserts are attached to a rod. In other embodiments, the inserts are attached to other elongated members, including fasteners, wires, and cables. The various different types of members may be constructed to be flexible or inflexible, and include a variety of different cross-sectional shapes. Member embodiments may be solid, or may include a hollow interior. Embodiments may further include monofilament and single strand wire along with multi-filament and multi-strand cable and ropes. Specific embodiments include cables used for securing together vertebral members. Another specific embodiment includes a shaft of a screw.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. In one embodiment, the inserts are integrally formed with the elongated members. The inserts may be fixedly positioned or movable along the elongated members. The present embodiments are, therefore, to be considered in all respects as illustrative and not restrictive, and all changes coming within the meaning and equivalency range of the appended claims are intended to be embraced therein.

What is claimed is:

1. A spinal fixation device comprising:
a receiver having a body and a channel formed in the body and adapted to receive one of a set of interchangeable inserts, said interchangeable inserts engage elongated members of different sizes;
a bone anchor to secure the receiver to a vertebral member.

2. The spinal fixation device of claim 1 wherein said insert includes a retaining feature to retain the insert within the channel of the receiver.

3. The spinal fixation device of claim 2 wherein the retaining feature comprises a clip to engage a sidewall of said channel.

4. The spinal fixation device of claim 1 wherein said insert includes an orientation feature to orient the insert in a predetermined orientation within the channel.

5. The spinal fixation device of claim 4 wherein said orientation feature comprises a flat formed in an outer surface of the insert.

6. The spinal fixation device of claim 1 further including a locking element to lock the insert within the channel.

7. The spinal fixation device of claim 6 further wherein the locking element comprises a locking screw that engages mating threads on the inner surface of the channel and applies compressive forces to the insert.

8. The spinal fixation device of claim 1 wherein said insert deforms responsive to compressive forces to engage the elongated member.

9. The spinal fixation device of claim 1 wherein the insert and the elongated member have congruent shapes.

10. The spinal fixation device of claim 1 wherein the insert and the elongated member have non-congruent shapes.

11. The spinal fixation device of claim 1 wherein the bone anchor comprises a multi-axial screw that allows relative angular adjustment between the receiver and the multi-axial screw.

12. The spinal fixation device of claim 1 wherein said inserts are integrally formed with elongated members of different sizes.

13. The spinal fixation device of claim 1 wherein the insert comprises an outer layer made of a first material and an inner layer made of a second material.

14. The spinal fixation device of claim 1 wherein said receiver and said bone anchor are integrally formed.

15. A spinal fixation device comprising:
a receiver having a body with a channel formed therein;
an insert adapted to be received within the channel, said insert having an outer layer made of a first material and an inner layer made of a second material; and
a bone anchor to secure the receiver to a vertebral member.

16. The spinal fixation device of claim 15 wherein said insert includes a retaining feature to retain the insert within the channel of the receiver.

17. The spinal fixation device of claim 16 wherein the retaining feature comprises a clip to engage a sidewall of said channel.

18. The spinal fixation device of claim 15 wherein said insert includes an orientation feature to orient the insert in a predetermined orientation within the channel.

19. The spinal fixation device of claim 18 wherein said orientation feature comprises a flat formed in an outer surface of the insert.

20. The spinal fixation device of claim 15 further including a locking element to lock the insert within the channel.

21. The spinal fixation device of claim 20 further wherein the locking element comprises a locking screw that engages mating threads on the inner surface of the channel and applies compressive forces to the insert.

22. A spinal fixation device comprising:
a receiver having a body with a channel formed therein;
an insert adapted to be received within the channel, said insert including an orientation feature to orient the insert in a predetermined orientation; and
a bone anchor to secure the receiver to a vertebral member.

23. The spinal fixation device of claim 22 wherein said orientation feature comprises a flat formed in an outer surface of the insert.

24. The spinal fixation device of claim 22 wherein said insert includes a retaining feature to retain the insert within the channel of the receiver.

25. The spinal fixation device of claim 24 wherein the retaining feature comprises a clip to engage a sidewall of said channel.

26. The spinal fixation device of claim 22 further including a locking element to lock the insert within the channel.

27. The spinal fixation device of claim 26 further wherein the locking element comprises a locking screw that engages mating threads on the inner surface of the channel and applies compressive forces to the insert.

28. The spinal fixation device of claim 22 wherein said orientation feature aligns a gap in the insert with an opening in the channel.

29. A method of securing a elongated member to a vertebral member, said method comprising:
anchoring a receiver including a body with a channel formed therein to said vertebral member;
selecting a elongated member to be secured to the vertebral member;
selecting an insert from a set of interchangeable inserts configured to fit the selected elongated member; and
disposing the insert around the selected elongated member; and
inserting the elongated member into the channel of the receiver.

30. The method of claim 29 further comprising orienting the insert within the channel in a predetermined orientation.

31. The method of claim 29 further comprising securing the insert against longitudinal movement within the channel by engaging a retaining feature on the insert with the body of the receiver.

32. The method of claim 29 wherein the insert comprises a composite insert having an outer portion and an inner portion made of different materials.
